(19) United States
(12) Patent Application Publication
(10) Pub. No.: US 2024/0041474 A1
(43) Pub. Date: Feb. 8, 2024

(54) Surgical Cutting Tool Having A Plurality Of Protuberances And Method Of Manufacturing The Same

(71) Applicant: Stryker European Operations Limited, Carrigtwohill, Co Cork (IE)

(72) Inventor: David Falvey, Carrigtwohill, Co. Cork (IE)

(73) Assignee: Stryker European Operations Limited, Carrigtwohill, Co Cork (IE)

(21) Appl. No.: 18/257,019

(22) PCT Filed: Dec. 13, 2021
(86) PCT No.: PCT/IB2021/061661

§ 371 Date: Jun. 12, 2023

Related U.S. Application Data
(60) Provisional application No. 63/124,453, filed on Dec. 11, 2020.

(51) Int. Cl. A61B 17/16

(52) U.S. Cl. CPC A61B 17/1615; A61B 2017/00526

(57) ABSTRACT

A surgical cutting bur includes a shaft extending between proximal and distal ends. A cutting head is coupled to the distal end of the shaft. The cutting head includes a body including an outer surface having an arcuate configuration and a plurality of protuberances each extending outwardly from the body. The protuberances are configured to engage and remove material when the surgical cutting bur is rotated by a powered surgical handpiece. Each of the plurality of protuberances defines at least one cutting edge. A method of manufacturing the surgical cutting bur includes the steps of sintering a powdered metal alloy and forming the body with the plurality of protuberances extending therefrom.

### CROSS-REFERENCE TO RELATED APPLICATIONS

The subject patent application claims priority to, and all the benefits of, United States Provisional Patent Application No. 63/124,453, filed on Dec. 11, 2020, the entire contents of which are incorporated by reference herein.

### SUMMARY

In one aspect a method of manufacturing a cutting tool or a high speed surgical drill is disclosed. The method comprising the steps of providing a powdered metal alloy; sintering the powdered metal alloy and forming a unitary body portion with a plurality of protuberances extending therefrom. The unitary body portion comprises a coupling region defining an interior. The method further comprises a step of removing unsintered powdered metal alloy from the interior. The method further comprises a step of positioning a drive shaft at least partially within the interior. The method further comprises a step of coupling the drive shaft to the unitary body portion.

In another aspect a surgical cutting tool for use with a powered surgical handpiece is disclosed. The surgical cutting tool comprises a shaft extending longitudinally between a proximal end configured to be coupled to the powered surgical handpiece and a distal end. The surgical cutting tool further comprises a cutting head coupled to the shaft at the distal end. The cutting head comprises a body comprising an outer surface having an arcuate configuration; and a plurality of protuberances each extending outwardly from the body and configured to engage and remove material when the surgical cutting tool is rotated by the powered surgical handpiece, each of the plurality of protuberance defining at least one cutting edge, and wherein the cutting head is free of cutting flutes.

In another aspect a method of using a high speed drill on a patient is disclosed. The high speed drill includes a body defining an outer surface having an arcuate configuration, and a plurality of protuberances extending from the body, wherein the protuberances are integral with and formed from the same material as the body. The protuberances extend from the body at a distance ranging from 0.1 to 0.5 millimeters. The method comprises rotating the body at a speed in excess of 60,000 rpm; and contacting bone and/or cartilage adjacent a critical structure with at least one of the plurality of protuberances. A critical structure may include sensitive tissue that may be easily damaged, slow to heal, or support function of other structures. For example, critical structures may include soft tissue, circulatory tissue (e.g. vessels), nerves, and other functionally-critical tissue.

Any of the above aspects can be combined in full or in part. Any features of the above aspects can be combined in full or in part. Any of the above implementations for any aspect can be combined with any other aspect. Any of the above implementations can be combined with any other implementation whether for the same aspect or a different aspect.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 is a perspective view of a surgical cutting tool coupled to a handpiece.

FIG. 2 is a front-side perspective view of a first implementation of a surgical cutting tool comprising a shaft and a cutting head, and showing a plurality of protuberances.

FIG. 3 is a close-up cross-sectional view of a distal end of the surgical cutting tool of FIG. 2 showing the shaft and the cutting head.

FIG. 4 is a partial rear-side exploded view of the surgical cutting tool of FIG. 2 with the shaft spaced from the cutting head.

FIG. 5 is a close-up perspective view of the cutting head of FIG. 4.

FIG. 6 is a close-up cross-sectional view of the cutting head taken along line 6-6 of FIG. 5.

FIG. 7 is a close-up cross-sectional view of the cutting head taken along line 7-7 of FIG. 3.

FIG. 8 is a front-side perspective view of a second implementation of a surgical cutting tool comprising a shaft and a cutting head, and showing a plurality of protuberances.

FIG. 9 is a close-up side view of a distal end of the surgical cutting tool of FIG. 8.

FIG. 10 is a partial rear-side perspective view of the surgical cutting tool of FIG. 8.

FIG. 11 is a close-up cross-sectional view of the cutting head taken along line 11-11 of FIG. 10.

FIG. 12 is a close-up cross-sectional view of the cutting head taken along line 12-12 of FIG. 9.

FIG. 13 is a close-up cross-sectional view of the cutting head taken along line 13-13 of FIG. 9.

### DETAILED DESCRIPTION

Surgical cutting tools are utilized by medical practitioners during surgical procedures. Some types of surgical cutting tools are commonly rotated by a powered surgical handpiece and may be configured for abrading and/or resecting bone and other tissue. In one example, the surgical cutting tool may include a bur having a cutting head with course grit diamonds bonded thereto to form an abrasive surface. As the surgical cutting tool is rotated the course grit diamonds rub against the bone and remove material. However, if the diamonds are not properly bonded to the cutting head, the diamonds may fall off the cutting head. Furthermore, the diamonds often vary in a size, shape, and placement on the cutting head, causing variances in the cutting profile of the surgical cutting tool as well as the removal rate of the material. Furthermore, the removed material is prone to becoming stuck to the cutting head and clogged between the diamonds. Other surgical cutting tools may utilize metallic teeth for material removal. However, these teeth do not have the physical properties of the diamonds that produce the desired abrasion and resection properties of diamonds. Furthermore, it is difficult (if not impossible) to produce a metallic surgical cutting tool with the physical properties of diamonds using commonly utilized subtractive manufacturing processes. As such, a surgical cutting tool that overcomes these challenges is desired.

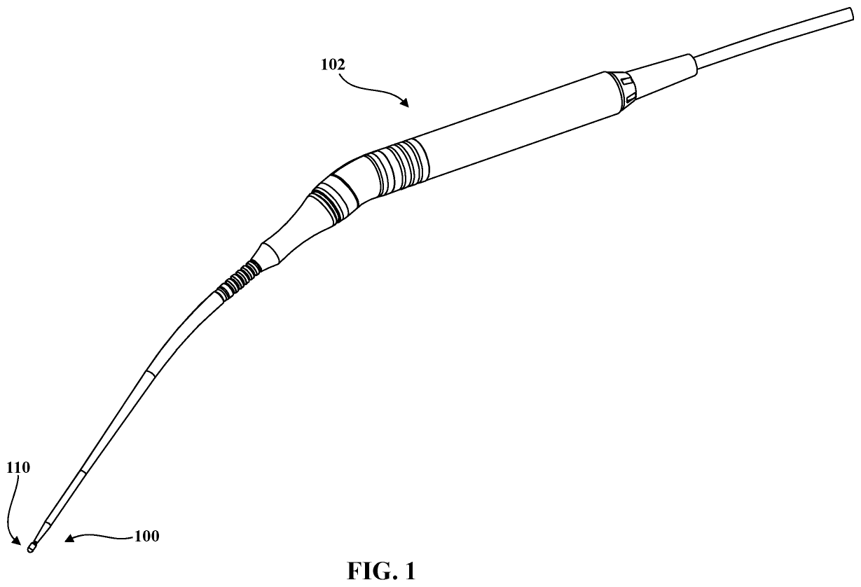

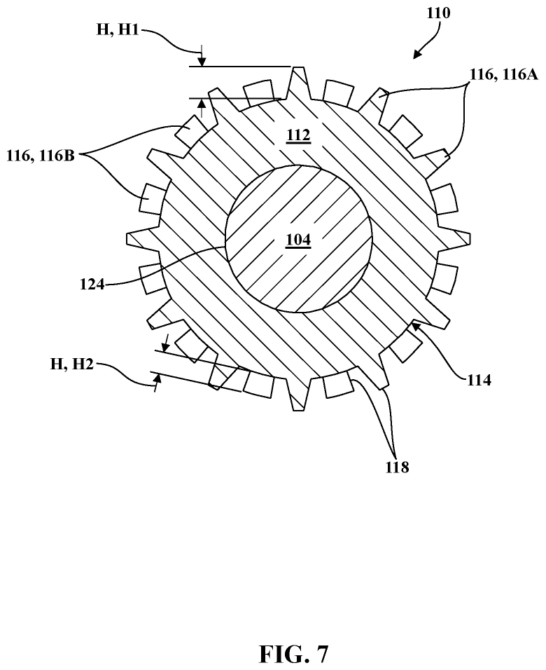

Referring to FIG. 1, a surgical cutting tool is shown at 100 for use with an exemplary powered surgical handpiece 102. The surgical cutting tool 100 as shown throughout Figures and described below may be used in a medical procedure for a patient (not shown). In one implementation the surgical cutting tool 100 may be configured as a bur, such as shown in FIGS. 1-13. In another implementation the surgical cutting tool may be configured as a hollow tube for use with a surgical shaver (not shown). In yet another implementation, the surgical cutting tool may be further configured as a saw blade (not shown). The surgical handpiece 102 may have a motor that drives the surgical cutting tool 100 at a high rate of speed, including rotational and/or oscillatory motion. For example, the surgical cutting tool 100 may be rotated at rates exceeding 10,000 rpm. In some implementations the surgical cutting tool 100 may be rotated in excess of rpm, in excess of 70,000 rpm, and in excess of 80,000 rpm. Exemplary uses of the surgical cutting tool 100 include abrading and resecting bone and other tissue, such as during endoscopic sinus surgery. However, the surgical cutting tool 100 may also be adapted for other medical procedures, including but not limited to, spinal, cranial, and endoscopic applications. It should be appreciated that the surgical cutting tool 100 may be operated by a user such as a surgeon.

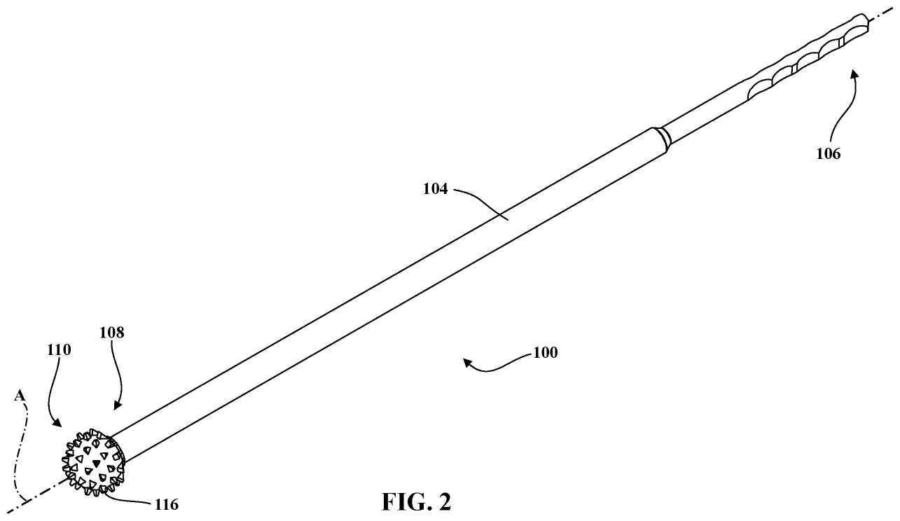

Turning now to FIGS. 2-7, a first implementation of the surgical cutting tool 100 is shown. Here, the surgical cutting tool 100 comprises a shaft 104 extending longitudinally along a longitudinal axis A between a proximal end 106 and a distal end 108. The shaft 104 is configured to be coupled with the powered surgical handpiece 102 at the proximal end 106. The surgical cutting tool 100 further comprises a cutting head 110 coupled to the distal end 108 of the shaft 104. The cutting head 110 comprises a body 112 comprising an outer surface 114 having an arcuate configuration. In the implementation of the surgical cutting tool 100 shown herein the body 112 may have a unitary construction. The arcuate configuration of the cutting head 110 may further be defined as spherical or semi-spherical. The cutting head 110 may further comprise a plurality of protuberances 116, alternatively referred to as teeth. Each of the plurality of protuberances 116 extends outwardly from the outer surface 114 of the body 112.

The plurality of protuberances 116 are configured to remove material when the surgical cutting tool 100 is rotated by the powered surgical handpiece 102 and the cutting head 110 is engaged with bone. The protuberances 116 may be uniformly distributed about the outer surface 114 to facilitate even and consistent removal of material when rotated by the powered surgical handpiece 102. The plurality of protuberances 116 may further be distributed about a portion, or substantially all, of the outer surface 114. Each protuberance 116 of the plurality of protuberances 116 may define at least one cutting edge 118.

During use the motor transfers torque to the shaft 104, which in-turn rotates and/or oscillates the cutting head 110 disposed at the distal end 108 of the shaft 104. The motor may rotate the shaft 104 and the cutting head 110 at speeds greater than 50,000 rpm. The high-speed rotation and torque transfer from the motor to the cutting head 110 allows the surgical cutting tool 100 to accurately and efficiently abrade or resect the bone and tissue during the surgical procedure as described above. Although the cutting head 110 is configured to be rotated by the shaft 104s, the cutting head 110 may be configured for non-rotatable motion (such as reciprocation) in other implementations of the surgical cutting tool.

As described above, the body 112 of the cutting head 110 comprises the outer surface 114 having the arcuate configuration. In the examples shown in FIGS. 2-7, the arcuate configuration may be further defined as a substantially spherical configuration. The spherical configuration of the body 112 facilitates the disposition of the protrusions (and the associated cutting edges 118) almost entirely around the cutting head 110. As such, the spherical body 112 allows the cutting head 110 to contact and remove material moved in almost any direction and orientation. Any arcuate configuration may be utilized for the outer surface 114 of the body 112, including (but not limited to) a hemispherical shape, a paraboloid shape, acorn-shape, a cone shape, and a cylinder shape (barrel-shaped). In other words, the cutting head may include at least one arcuate surface.

Turning now to the cross-sectional view of FIG. 3, the distal end 108 of the surgical cutting tool 100 is shown. This view shows the cutting head 110 and the plurality of protuberances 116 as well as engagement between the shaft 104 and the body 112. In addition to the arcuate configuration, the body 112 may comprise a coupling region 122 defining an interior 124. The coupling region 122 is generally positioned on a proximally directed portion of the cutting head 110 such that the interior 124 is open at a proximal end of the cutting head 110. The interior 124 is configured to receive the distal end 108 of the shaft 104 to form the surgical cutting tool 100. Engagement between an outer surface of the shaft 104 and an inner surface of the interior 124 transfers rotation from the shaft 104 to the cutting head 110 to effect material removal when the protuberances 116 are engaged with the bone.

The distal end 108 of the shaft 104 and the interior 124 of the coupling region 122 may be coupled during manufacturing such that the shaft 104 and the cutting head 110 are rotationally fixed. The cutting head 110 is also axially fixed to the distal end 108 of the shaft 104 during manufacturing. The distal end 108 and the interior 124 are configured with corresponding shapes, shown here as circular. To this end, the illustrated implementation of the cutting tool 100 employs a manufacturing step of bonding the shaft 104 to the cutting head 110. Specifically, the distal end 108 of the shaft 104 is positioned in the interior 124 and may be joined with a brazing process (i.e. brazed together), which axially and rotationally fixes the shaft 104 and the cutting head 110. Alternative bonding processes may be utilized, for example, welding, friction welding, plug welding, soldering, gluing, and the like. Other configurations of the distal end 108 and the coupling region 122 may enable additional alternative coupling methods to be utilized. For example, the shaft and the interior may have a hexagonal configuration to prevent relative rotation. Further alternative configurations include triangular, square, splined, multifaceted, D-shaped, etc. An exemplary alternative coupling method that may be utilized is an interference fit. In other implementations of the cutting tool (not shown) the distal end of the shaft and the interior of the coupling region may be formed with corresponding threads and coupled using a threaded engagement.

The shaft 104 may be manufactured from a material suitable for transferring torque from the motor of the handpiece 102 to the cutting head 110. The shaft 104 may be formed from a material that is different from the material of the cutting head 110. For example, the shaft 104 may comprise a steel alloy, such as stainless-steel and alloys thereof. Additionally, the shaft 104 may comprise titanium and alloys thereof. In one example, the shaft 104 may be formed from stainless-steel and the cutting head 110 may be constructed from titanium alloy. In another example, the shaft 104 may be formed from a titanium alloy and the cutting head 110 may be formed from stainless-steel. Some implementations of the shaft may be manufactured from a material that contains no nickel, or is free of nickel. Additional materials such as composites or shape-memory alloys are also contemplated.

In the implementation shown here, the plurality of protuberances 116 may extend a uniform height H from the outer surface 114. Each of the protuberances 116 shown in FIG. 3 extending from the surface 114 substantially the same distance to the height H. The uniformity of the height H facilitates the protuberances 116 evenly removing material as the surgical cutting tool 100 rotates and produces a substantially even surface along the material following removal. Exemplary values of the uniform height H may be approximately 1/10 the diameter of the body 112, and more specifically between 0.05 millimeters to 2.0 millimeters. Other exemplary values of the uniform height H may range from approximately 0.1 mm to 0.5 mm. Said differently, the plurality of the protuberances 116 may extend from the body 112 by a distance between 0.1 mm and 0.5 mm. In other implementations utilizing a larger body 112, the uniform height H may be greater than 0.5 mm, such as, for example, when the body 112 has a diameter of 10 mm the protuberances 116 may extend a distance of approximately 1.0 mm. In other implementations the uniform height H may range from approximately 0.1 mm to 0.3 mm. In yet another implementation the uniform height H may be approximately 0.2 mm or approximately 0.5 mm.

In some instances the uniform height H may refer to differing lengths of the protuberances 116 in which the protuberances 116 form a pattern or a defined cutting profile along tips of the protuberances 116. For example, the protuberances 116 may comprise at least a first set protuberances 116A each extending a first uniform height H1 from the outer surface 114 and a second set of protuberances 116B each extending a second uniform height H2 from the outer surface 114, with the first uniform height H1 differing from the second uniform height H2. More specifically, the second set of protuberances 116B may be disposed on a side of the body 112 that faces the shaft 104 while the first set of protuberances 116A may be disposed on an opposing side of the body 112 that faces away from the shaft 104. The difference in the first and second uniform heights H1, H2 facilitates differences in the removal of material when the surgical cutting tool 100 is pushed into the material in comparison to the surgical cutting tool 100 being pulled out of the material. Similarly, the heights H1, H2 could be based on angle relative to the shaft 104 such that the operator can vary the material removal rate, achieved through different protuberance heights, by tilting the surgical cutting tool to engage the tissue with a different portion of the cutting head. In certain aspects, each set of protuberances of the cutting head may include at least five protuberances. As with above, the first and second sets of protuberances 116A, 116B may be implemented in various forms and configurations. As such, exemplary values of the uniform heights H1, H2 may range from 0.05 mm and 2.0 mm. Other exemplary values of the uniform heights H1, H2 may range from approximately 0.1 mm to 0.5 mm. In other implementations the uniform heights H1, H2 may range from approximately 0.1 mm to 0.3 mm. In one exemplary implementation the first uniform height H1 may be approximately 0.5 mm and the second uniform height H1 approximately 0.2 mm. Other distances and heights are contemplated.

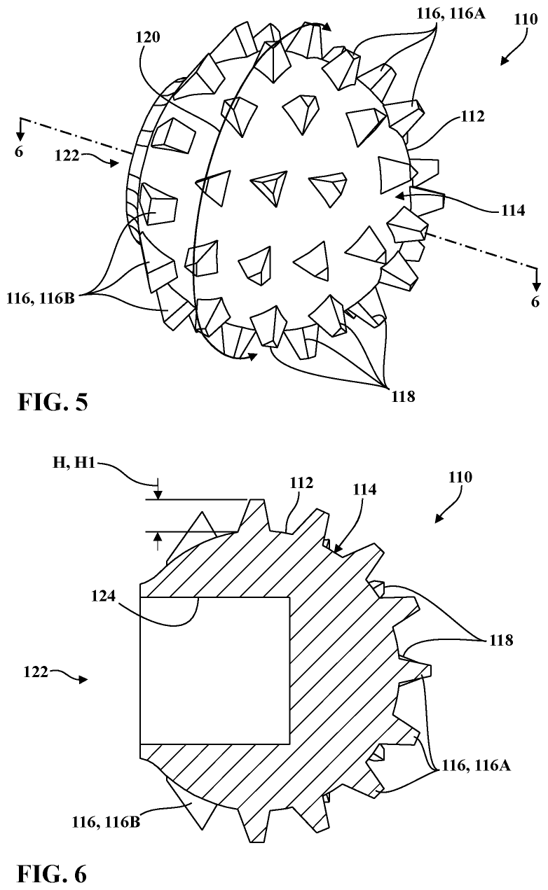

In other embodiments the one or more sets of protuberances 116 may be uniformly spaced from one another along the entire outer surface 114. For example, the uniform spacing refers to the protuberances 116 being substantially equidistant from one another along the outer surface 114, as shown in FIG. 5. The space between the protuberances 116 acts as a gutter to receive material removed by the protuberances 116 and carry the removed material away from the cut site. The space between the protuberances 116 is distinguished from a flute in that material cut by each cutting edge 118 is not directed to a particular flute but rather into the various spaces surrounding each protuberance 116. As such, the cutting head 110 may be free from a flute. The rotation of the cutting head 110 subsequently ejects the material through inertia preventing the cutting head 110 from becoming clogged. As shown in FIG. 6, the protuberances 116 may further be aligned in at least one row 120, with the row 120 radially arranged along the body 112 of the cutting head 110. The at least one row 120 may further be defined as a plurality of rows 120 spaced from one another and radially arranged along the body 112. In some implementations the at least one row 120 may be helically disposed along the body 112. The spacing between the rows 120, in conjunction with the helical configuration of the rows 120, channels material removed by the protuberances 116 away from the cut site and toward the shaft 104, where the material is ejected through inertia.

In other implementations, each of the protuberances 116 may further have an angled configuration and extend from the arcuately configured outer surface 114 of the body 112. The angled configuration of the protuberances 116 extending from the arcuate configuration of the cutting head is particularly difficult to produce using subtractive manufacturing. The additive and sintering processes described herein are suited for producing the compound configurations described for certain exemplary cutting heads.

In other implementations, each of the protuberances 116 may alternatively have an arcuate configuration and extend from the arcuately configured outer surface 114 of the body 112. The arcuate configuration of the protuberances extending from the other arcuate configuration (of the cutting head) is particularly difficult to produce using subtractive manufacturing. The additive and sintering processes described above are suited for producing the compound arcuate configurations described for certain exemplary cutting heads.

In the implementation of the surgical cutting tool 100 shown in FIG. 5, some of the protuberances 116 have an angled and tapered configuration. Here, each of the protuberances 116 tapers, or reduces in cross-sectional area, as it extends from the outer surface 114 to the height H. Additionally, the protuberances 116 may be truncated at a distal surface resulting in a frustopyramidal configuration. Said differently, the protuberances 116 may be configured as truncated tetrahedrons, which have a triangular cross-sectional shape. Alternatively, the protuberances 116 may taper to a point.

Each of the protuberances 116 defines at least one cutting edge 118 where adjacent faces meet. Here, each protuberance 116 defines six cutting edges 118. The angle formed at the cutting edge 118 makes the protuberance 116 capable of cutting and/or abrading the material when the surgical cutting tool 100 is rotated and engaged with the material. The protuberances may be configured with alternative cross-sectional shapes such as, for example, circular, square, rectangular, cross-shaped, multifaceted, etc. More specifically, protuberances with a circular cross-sectional shape may result in a conical configuration or frustoconical shape having a single circular cutting edge. In a further example the protuberances may be configured as a curved rectangular prism, with each protuberance defining eight cutting edges.

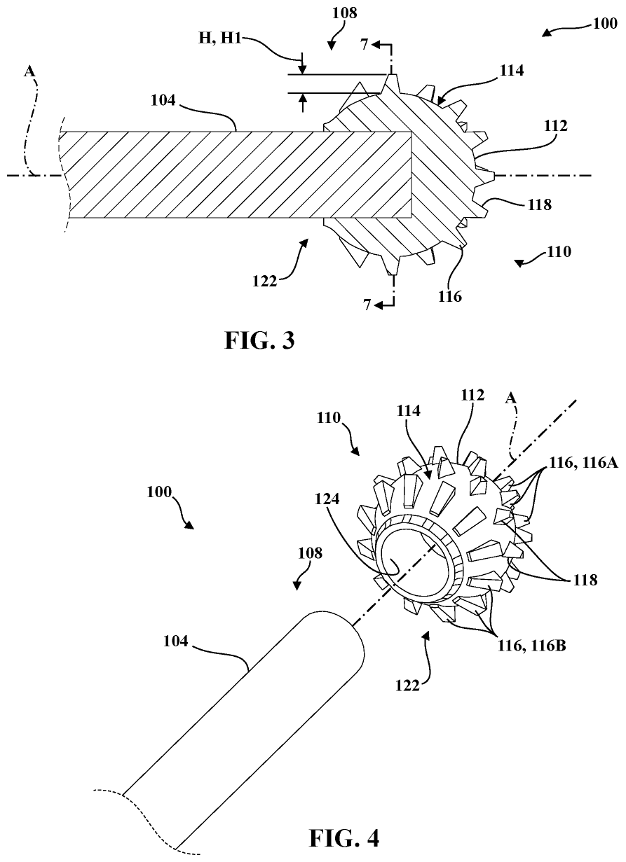

Each of the protuberances 116 may be disposed about the outer surface 114 of the body 112 at varying angles relative to one another. More specifically, as shown in FIGS. 4 and 5, the arcuate configuration of the outer surface 114 causes the protuberances 116 (which extend outwardly and substantially orthogonal to the outer surface 114) to be angled relative to one another. The varying angles present the cutting surface of the protuberances 116 varying angles of attack relative to the material. Therefore, the abrasive properties of each protuberance 116 can vary depending on the angle of the protuberance 116, allowing the cutting properties of the cutting head 110 to be varied by changing the angles of the protuberances 116.

Shown in FIGS. 2-7, the plurality of protuberances 116 on the cutting head 110 may be further defined as two sets, a first set of protuberances 116A and a second set of protuberances 116B. The first set of protuberances 116A may be arranged about a distal hemisphere of the body 112 and the second set of protuberances 116B may radially arranged about the longitudinal axis adjacent to the coupling region 122. Each protuberance in the respective set of protuberances has a substantially similar configuration. Said differently, each of the first set of protuberances 116A are substantially uniform has generally the same size (i.e., length, width, and depth) and shape. Likewise, each of the second set of protuberances 116B are substantially uniform has generally the same size (i.e., length, width, and depth) and shape. However, in other examples (not shown) the size and/or the shape of the protuberances 116 may vary along the outer surface 114 to produce a different cutting profile or cutting characteristics. The configuration of the first set of protuberances 116A may differ from the configuration of the second set of protuberances 116B. Specifically, each of the first set of protuberances 116A is generally configured as a truncated pyramid with a triangular cross-section while each of the second set of protuberances 116B is generally configured as a prism with a rectangular cross-section. Additionally, the first set of protuberances 116A may extend from the outer surface 114 to a uniform height different than the uniform height of the second set of protuberances 116B. More specifically, the first set of protuberances 116A may extend a first uniform height H1 and the second set of protuberances 116B may extend a second uniform height H2, with the first uniform height H1 differing from the second uniform height H2.

The plurality of protuberances 116 may be further defined as at least ten protuberances 116. Further, the plurality of protuberances 116 may be further defined as at least twenty protuberances 116. Further still, the plurality of protuberances 116 may be further defined as at least fifty protuberances 116. It should be appreciated that any suitable number of protuberances 116 may be utilized according to the relative size and spacing of the protuberances 116 and the size of the cutting head 110. Furthermore, the at least one cutting edge 118 is further defined as at least fifty distinct cutting edges 118. The at least fifty distinct cutting edges 118 may be disposed individually on fifty protuberances 116. Alternatively, the at least fifty distinct cutting edges 118 may be disposed in any suitable numbers on less than fifty protuberances 116 (i.e., more than one cutting edge 118 per protuberance 116). More specifically, each distinct cutting edge 118 may correspond to a geometric edge of the protuberance 116 where multiple faces meet at an angle. More specifically, and for example, the tetrahedral configuration of the protuberances 116 may comprise three distinct cutting edges 118 where each protruding face meets another protruding face. Alternatively, when the protuberances 116 are configured as frustopyramidal, each protuberance 116 may comprise six distinct cutting edges 118: three cutting edges protruding at an angle to the outer surface 114 defining angled side faces of each protuberance 116, and a further three cutting edges approximately parallel to the outer surface 114 that define a distal end face of each protuberance 116.

In addition to the body 112 having a unitary construction the plurality of protuberances 116 may be integrally formed and unitary with the body 112. Said differently, the body 112 and the plurality of protuberance 116 may be formed of a single material. Said differently, the plurality of protuberances 116 may be affixed to the body as a singular component without the use of conventional coupling methods such as welding, adhesives, or fasteners. Moreover, the body 112 and the plurality of protuberances 116 are fused together, and each comprises a metallic material. In one example, the body 112 and the plurality of protuberances 116 are integrally formed from deposited layers of a metallic material. The deposition of layers of a material to form a solid component is referred to in the art by many names, but most commonly as additive manufacturing, 3D printing, or rapid prototyping. The metallic material may comprise a variety of metals in various ratios with other non-metals. In some implementations the metallic material may be a metal alloy. More specifically, each of the deposited layers may comprise a metal alloy in a powdered form. In one implementation, the metallic material may comprise titanium and alloys thereof. Alternatively and in other implementations, the metallic material may comprise a stainless-steel alloy. Other materials are contemplated. Furthermore, the cutting tool, including the cutting head, may be free of nickel.

In another implementation, the body 112 and the plurality of protuberances 116 are integrally formed from deposited, compacted, and heated layers of a material, commonly referred to in the art as sintering. Here, the material may comprise a metallic material such as a carbide alloy and/or a ceramic. However, other materials capable of being formed into a solid component using a sintering process may be utilized. As with above, the body 112 and the protuberances 116 are additively formed by adding and solidifying the material, rather than removing material (commonly referred to subtractive manufacturing). Common subtractive manufacturing processes include cutting, milling, drilling, turning, etc. The formation of the body 112 and the protuberances 116 by adding material allows for the formation of shapes and geometries that are incapable of being formed by subtractive manufacturing (or less efficiently formed by subtractive manufacturing).

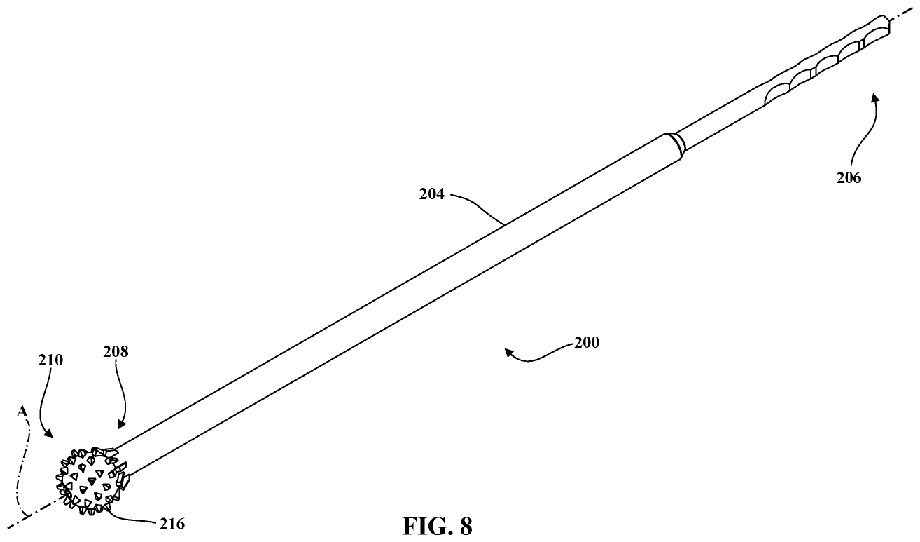

Turning now to FIGS. 8-13, another version of the surgical cutting tool is shown. As will be appreciated from the subsequent description below, the second surgical cutting tool is similar to the surgical cutting tool 100 described above in connection with FIGS. 2-7. As such, the components and structural features of the second version of the surgical cutting tool 200 that are the same as, or that otherwise correspond to, the first version of the surgical cutting tool 100 are provided with reference numerals increased by 100 (e.g. 100 and 200). While the specific differences between these versions will be described in detail, for the purposes of clarity, consistency, and brevity, only certain structural features and components common between these versions will be discussed and depicted in the drawings of the second version of the surgical cutting tool 200. Here, unless otherwise indicated, the above description of the first version of the surgical cutting tool 100 may be incorporated by reference with respect to the second version of the surgical cutting tool 200 without limitation.

The second implementation of the surgical cutting tool 200 shown in FIGS. 8-13 comprises a shaft 204 extending longitudinally along a longitudinal axis A between a proximal end 206 and a distal end 208. The shaft 204 is configured to be coupled with the powered surgical handpiece 202 at the proximal end 206. The surgical cutting tool 200 further comprises a cutting head 210 coupled to the distal end 208 of the shaft 204. The cutting head 210 comprises a body 212 comprising an outer surface 214 having an arcuate configuration. In the implementation of the surgical cutting tool 200 shown herein the body 212 may have a unitary construction. The arcuate configuration of the cutting head 210 may further be defined as spherical or semi-spherical. The cutting head 210 may further comprise a plurality of protuberances 216, alternatively referred to as teeth. Each of the plurality of protuberances 216 extends outwardly from the outer surface 214 of the body 212. The plurality of protuberances 216 are configured to remove material when the surgical cutting tool 200 is rotated by the powered surgical handpiece 202 and the cutting head 210 is engaged with bone. The protuberances 216 may be uniformly distributed about the outer surface 214 to facilitate even and consistent removal of material when rotated by the powered surgical handpiece 202. The plurality of protuberances 216 may further be distributed about a portion, or substantially all, of the outer surface 214. Each protuberance 216 of the plurality of protuberances 216 may define at least one cutting edge 218.

As described above, the body 212 of the cutting head 210 comprises the outer surface 214 having the arcuate configuration. Similar to above the arcuate configuration may be further defined as a substantially spherical configuration. The spherical configuration of the body 212 facilitates the disposition of the protrusions (and the associated cutting edges 218) almost entirely around the cutting head 210. As such, the spherical body 212 allows the cutting head 210 to contact and remove material moved in almost any direction and orientation. Any arcuate configuration may be utilized for the outer surface 214 of the body 212, including (but not limited to) a hemispherical shape, a paraboloid shape, acorn-shape, a cone shape, and a cylinder shape (barrel-shaped). In other words, the cutting head may include at least one arcuate surface.

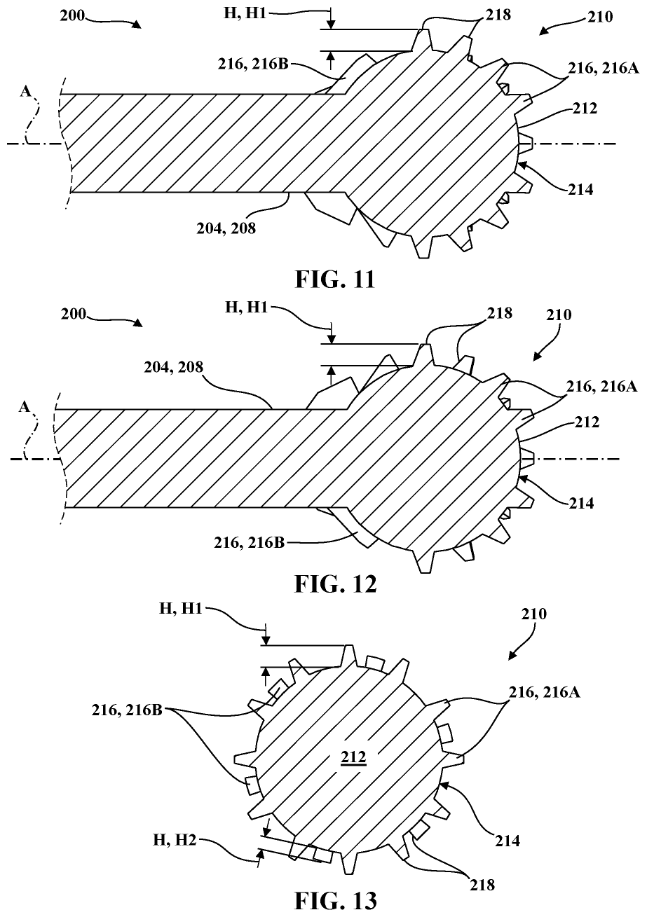

Turning now to the cross-sectional view of FIG. 11, the distal end 208 of the surgical cutting tool 200 is shown. This view shows the cutting head 210 and the plurality of protuberances 216. Here, the distal end 208 of the shaft 204 and the cutting head 210 may be integrally formed such that the shaft 204 and the cutting head 210 are rotationally and axially fixed. Said differently, the cutting head 210 and the shaft 204 are formed as a unitary structure from the same material. The cutting head 210 and the shaft 204 may be monolithic and formed simultaneously.

As above, the plurality of protuberances 216 may extend a uniform height H from the outer surface 214. Each of the protuberances 216 shown in FIG. 13 extending from the surface 214 substantially the same distance to the height H. However, uniform height H may refer to differing lengths of the protuberances 216 in which the protuberances 216 form a pattern or a defined cutting profile along tips of the protuberances 216. For example, the protuberances 216 may comprise at least a first set protuberances 216A each extending a first uniform height H1 from the outer surface 214 and a second set of protuberances 216B each extending a second uniform height H2 from the outer surface 214, with the first uniform height H1 differing from the second uniform height H2. More specifically, the second set of protuberances 216B may be disposed on a side of the body 212 that faces the shaft 204 while the first set of protuberances 216A may be disposed on an opposing side of the body 212 that faces away from the shaft 204. The difference in the first and second uniform heights H1, H2 facilitates differences in the removal of material when the surgical cutting tool 200 is pushed into the material in comparison to the surgical cutting tool 200 being pulled out of the material. Similarly, the heights could be based on angle relative to the shaft 204 such that the operator can vary the material removal rate, achieved through different protuberance heights, by tilting the surgical cutting tool to engage the tissue with a different portion of the cutting head. In certain aspects, each set of protuberances of the cutting head may include at least five protuberances.

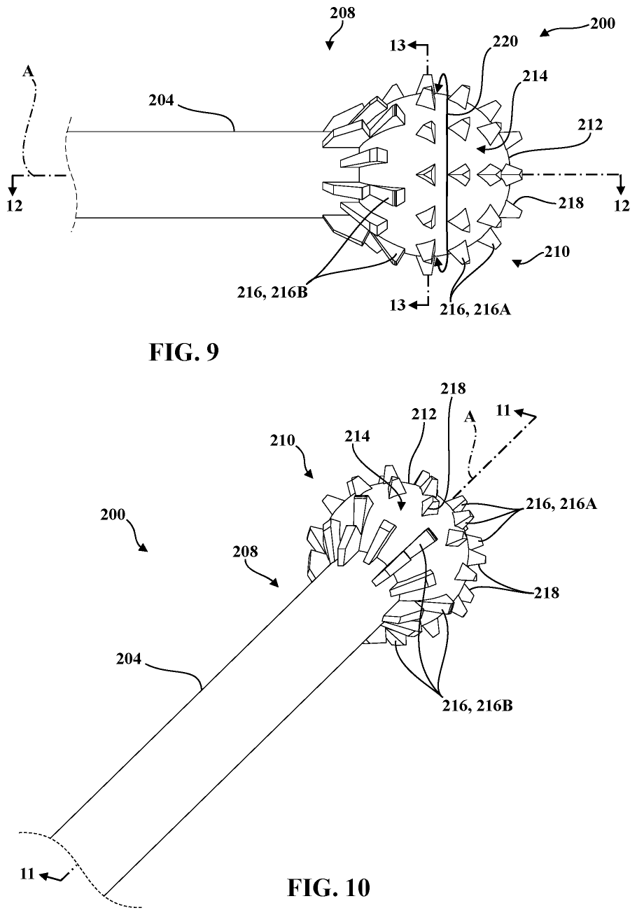

In other embodiments the one or more sets of protuberances 216 may be uniformly spaced from one another along the entire outer surface 214. For example, the uniform spacing refers to the protuberances 216 being substantially equidistant from one another along the outer surface 214, as shown in FIG. 10. The space between the protuberances 216 acts as a gutter to receive material removed by the protuberances 216 and carry the removed material away from the cut site. The space between the protuberances 216 is distinguished from a flute in that material cut by each cutting edge 218 is not directed to a particular flute but rather into the various spaces surrounding each protuberance 216. As such, the cutting head 210 may be free from a flute. The rotation of the cutting head 210 subsequently ejects the material through inertia preventing the cutting head 210 from becoming clogged. As shown in FIG. 9, the protuberances 216 may further be aligned in at least one row 220, with the row 220 radially arranged along the body 212 of the cutting head 210. The at least one row 220 may further be defined as a plurality of rows 220 spaced from one another and radially arranged along the body 212. In some implementations the at least one row 220 may be helically disposed along the body 212. The spacing between the rows 220, in conjunction with the helical configuration of the rows 220, channels material removed by the protuberances 216 away from the cut site and toward the shaft 204, where the material is ejected through inertia.

In other implementations, each of the protuberances 216 may further have an angled configuration and extend from the arcuately configured outer surface 214 of the body 212. The angled configuration of the protuberances 216 extending from the arcuate configuration of the cutting head is particularly difficult to produce using subtractive manufacturing. The additive and sintering processes described herein are suited for producing the compound configurations described for certain exemplary cutting heads.

Each of the protuberances 216 may be disposed about the outer surface 214 of the body 212 at varying angles relative to one another. More specifically, as shown in FIGS. 9-13, the arcuate configuration of the outer surface 214 causes the protuberances 216 (which extend outwardly and substantially orthogonal to the outer surface 214) to be angled relative to one another. The varying angles present the cutting surface of the protuberances 216 varying angles of attack relative to the material. Therefore, the abrasive properties of each protuberance 216 can vary depending on the angle of the protuberance 216, allowing the cutting properties of the cutting head 210 to be varied by changing the angles of the protuberances 216.

The plurality of protuberances 216 may be further defined as at least ten protuberances 216. Further, the plurality of protuberances 216 may be further defined as at least twenty protuberances 216. Further still, the plurality of protuberances 216 may be further defined as at least fifty protuberances 216. It should be appreciated that any suitable number of protuberances 216 may be utilized according to the relative size and spacing of the protuberances 216 and the size of the cutting head 210. Furthermore, the at least one cutting edge 218 is further defined as at least fifty distinct cutting edges 218. The at least fifty distinct cutting edges 218 may be disposed individually on fifty protuberances 216. Alternatively, the at least fifty distinct cutting edges 218 may be disposed in any suitable numbers on less than fifty protuberances 216 (i.e., more than one cutting edge 218 per protuberance 216).

In addition to the body 212 having a unitary construction the plurality of protuberances 216 may be integrally formed and unitary with the body 212. Said differently, the body 212 and the plurality of protuberance 216 may be formed of a single material. Said differently, the plurality of protuberances 216 may be affixed to the body as a singular component without the use of conventional coupling methods such as welding, adhesives, or fasteners. Moreover, the body 212 and the plurality of protuberances 216 are fused together, and each comprises a metallic material. In one example, the body 212 and the plurality of protuberances 216 are integrally formed from deposited layers of a metallic material. The deposition of layers of a material to form a solid component is referred to in the art by many names, but most commonly as additive manufacturing, 3D printing, or rapid prototyping. The metallic material may comprise a variety of metals in various ratios with other non-metals. In some implementations the metallic material may be a metal alloy. More specifically, each of the deposited layers may comprise a metal alloy in a powdered form. In one implementation, the metallic material may comprise titanium and alloys thereof. Alternatively and in other implementations, the metallic material may comprise a stainless-steel alloy. Other materials are contemplated. Furthermore, the cutting tool, including the cutting head, may be free of nickel.

In another implementation, the body 212 and the plurality of protuberances 216 are integrally formed from deposited, compacted, and heated layers of a material, commonly referred to in the art as sintering. Here, the material may comprise a metallic material such as a carbide alloy and/or a ceramic. However, other materials capable of being formed into a solid component using a sintering process may be utilized. As with above, the body 212 and the protuberances 216 are additively formed by adding and solidifying the material, rather than removing material (commonly referred to subtractive manufacturing). Common subtractive manufacturing processes include cutting, milling, drilling, turning, etc. The formation of the body 212 and the protuberances 216 by adding material allows for the formation of shapes and geometries that are incapable of being formed by subtractive manufacturing (or less efficiently formed by subtractive manufacturing).

A method of manufacturing the surgical cutting tool 100, 200 shown in FIGS. 2-7 is described below. The method comprises a first step of providing a powdered metal alloy as described above. The powdered metal alloy may comprise a stainless-steel alloy in some implementations. The powered metal alloy may comprise a titanium alloy in some implementations. The method of manufacturing may further comprise a step of selectively sintering the powdered metal alloy and forming the unitary body portion 112 with a plurality of protuberances 116 extending therefrom. The unitary body portion 112 may comprise the coupling region 122 defining the interior 124. The method of manufacturing may further comprise a step of removing unsintered powdered metal alloy from the interior 124. The method of manufacturing may further comprise a step of positioning the drive shaft 104 at least partially within the interior 124 and coupling the drive shaft 104 to the unitary body portion 112.

The step of coupling the drive shaft 104 to the unitary body portion 112 may be further defined as bonding the drive shaft 104 to the unitary body portion 112. As described above, bonding the driveshaft 104 to the body portion 112 may comprise a brazing process.

The method may further comprise the step of depositing layer upon layer the metallic material to form the shaft 204. As such, the cutting head 210 and the shaft 204 may be integrally formed, as shown in FIGS. 8-13. However, the shaft may be individually formed and assembled to the cutting head by, for example, welding or mechanical fastening.

As described above, the metallic material may comprise or be further defined as a titanium alloy. When the metallic material is the titanium alloy, the method may further comprise the step of depositing a titanium nitride coating on the titanium alloy. The application of the titanium nitride coating increases the stiffness of the cutting edge 118 of the protuberances 116, improving the abrasive qualities and the longevity of the cutting edge 118.

In other implementations, the cutting head 110 may utilize one or more coatings in addition, or in the alternative, to the titanium nitride coating described above. These coatings may be applied only when the cutting head 110 is manufactured from certain the base materials or may be applied to cutting heads that have been manufactured with any suitable material. Further, manufacturing the cutting head 110 may comprise applying one or more surface treatments to modify the properties of the outer surface 114 or the protuberances 116. The coating(s) or surface treatment(s) may be utilized to increase the biocompatibility, the hardness, and/or the durability of the material. Similarly the coating(s) or surface treatment(s) may be utilized to tailor the properties of the cutting head, such as the abrasiveness.

As described above, the metallic material may be further defined as a carbide alloy. When the metallic material is the carbide alloy, the method may further comprise the step of grinding the protuberances 116. The step of grinding the protuberances 116 sharpens and stiffens the cutting edge 118 of the protuberances 116, improving the abrasive qualities and the longevity of the cutting edge 118.

The step of depositing layer upon layer the metallic material to form the body 112 with the outer surface 114 having an arcuate configuration may be further defined as depositing layer upon layer the metallic material to form the body 112 with the outer surface 114 having a substantially spherical configuration. The substantially spherical configuration is described in detail above.

The step of depositing layer upon layer the metallic material to form the plurality of protuberance 116 of the cutting further comprises forming the plurality of protuberance 116 of the cutting head 110 extending the uniform height H from the outer surface 114 and uniformly spaced from one another along the entire outer surface 114. The step of depositing layer upon layer the metallic material to form the plurality of protuberance 116 of the cutting further comprises forming the plurality of protuberance 116 each having the uniform size and shape. The uniform height H, spacing, size and shape of the protuberances 116 are described in detail above.

Several instances have been discussed in the foregoing description. However, the aspects discussed herein are not intended to be exhaustive or limit the disclosure to any particular form. Various modifications to these aspects will be readily apparent to those skilled in the art, and the generic principles defined herein may be applied to other aspects without departing from the scope of the disclosure. The terminology that has been used is intended to be in the nature of words of description rather than of limitation. Many modifications and variations are possible in light of the above teachings and the disclosure may be practiced otherwise than as specifically described.

### CLAUSES

I. A method of manufacturing a cutting tool or a high speed surgical drill, the method comprising the steps of: providing a powdered metal alloy; sintering the powdered metal alloy and forming a unitary body portion with a plurality of protuberances extending therefrom, and, optionally, wherein the unitary body portion comprises a coupling region defining an interior; removing unsintered powdered metal alloy from the interior; positioning a drive shaft at least partially within the interior; and coupling the drive shaft to the unitary body portion.
II. The method of clause I, wherein the powdered metal alloy comprises stainless-steel.
III. The method of clause I, wherein the powdered metal alloy comprises titanium.
IV. The method of clause I, wherein the step of coupling the drive shaft to the unitary body portion is further defined as bonding the drive shaft to the unitary body portion.
V. The method of clause I, wherein bonding the drive shaft to the unitary body portion comprises a brazing process.
VI. A surgical cutting tool for use with a powered surgical handpiece, the surgical cutting tool comprising: a shaft extending longitudinally between a proximal end configured to be coupled to the powered surgical handpiece and a distal end; a cutting head coupled to the shaft at the distal end, the cutting head comprising: a body comprising an outer surface having an arcuate configuration; and a plurality of protuberances each extending outwardly from the body and configured to engage and remove material when the surgical cutting tool is rotated by the powered surgical handpiece, each of the plurality of protuberance defining at least one cutting edge, and wherein the cutting head is free of cutting flutes.
VII. The surgical cutting tool of clause VI, wherein the shaft is formed separately from the cutting head.
VIII. The surgical cutting tool of clause VII, wherein the shaft is formed from a different material than the cutting head.
IX. The surgical cutting tool of clause VI, wherein each of the plurality of protuberances extend a uniform height from the outer surface.
X. The surgical cutting tool of clause VI, wherein the each of the plurality of protuberances is arranged uniformly spaced from one another about the outer surface.
XI. The surgical cutting tool of clause VI, wherein the plurality of protuberances comprises at least a first set protuberances each extending a first uniform height from the outer surface and a second set of protuberances each extending a second uniform height from the outer surface, with the first uniform height differing from the second uniform height.
XII. The surgical cutting tool of clause VI, wherein the plurality of protuberances is aligned in at least one row, with the at least one row radially arranged along the body of the cutting head.
XIII. The surgical cutting tool of clause VI, wherein the outer surface of the body of the cutting head has an arcuate configuration.
XIV. The surgical cutting tool of clause VI, wherein the plurality of protuberances has an angled configuration and extends from the arcuately configured outer surface of the body.
XV. The surgical cutting tool of clause XIV, wherein the angled configuration of the plurality of protuberances is further defined as a frustopyramidal configuration.
XVI. The surgical cutting tool of clause VI, wherein each of the plurality of protuberances has a uniform size and shape.
XVII. The surgical cutting tool of clause VI, wherein each of the plurality of protuberances is disposed along the body at varying angles relative to each another.
XVIII. The surgical cutting tool of clause VI, wherein the body and the plurality of protuberances are integrally formed.
XIX. The surgical cutting tool of clause XVIII, wherein the body and the plurality of protuberances are fused together, and each comprises a metallic material.
XX. The surgical cutting tool of clause XIX, wherein the body and the plurality of protuberances are integrally formed from deposited layers of the metallic material.
XXI. The surgical cutting tool of clause XIX, wherein the body and the plurality of protuberances are integrally formed from deposited, compacted, and heated layers of the metallic material.
XXII. The surgical cutting tool of clause XXI, wherein the metallic material comprises a carbide alloy.
XXIII. The surgical cutting tool of clause XIX, wherein the metallic material comprises a titanium alloy.
XXIV. The surgical cutting tool of clause XIX, wherein the metallic material comprises a stainless-steel alloy.
XXV. The surgical cutting tool of clause VI, wherein the cutting head is free of nickel.
XXVI. The surgical cutting tool of clause VI, wherein the plurality of protuberances is further defined as at least ten protuberances.
XXVII. The surgical cutting tool of clause XXVI, wherein the plurality of protuberances is further defined as at least twenty protuberances.
XXVIII. The surgical cutting tool of clause XXVII, wherein the plurality of protuberances is further defined as at least fifty protuberances.
XXIX. The surgical cutting tool of clause VI, wherein the at least one cutting edge is further defined as at least fifty distinct cutting edges.
XXX. A method of using a high speed drill on a patient, the high speed drill including a body defining an outer surface having an arcuate configuration, and a plurality of protuberances extending from the body, wherein the protuberances are integral with and formed from the same material as the body, and extending from the body at a distance ranging from 0.1 to 0.5 millimeters, the method comprising: rotating the body at a speed in excess of 60,000 rpm; and contacting bone and/or cartilage adjacent a critical structure with at least one of the plurality of protuberances.
XXXI. A surgical cutting tool for use with a powered surgical handpiece, said surgical cutting tool comprising: a shaft extending longitudinally between a proximal end and a distal end, with said shaft configured to be coupled with the powered surgical handpiece at said proximal end; a cutting head mounted to said shaft at said distal end, with said cutting head comprising: a body comprising an outer surface having an arcuate configuration; and a plurality of protuberance each extending outwardly from said body and configured to engage and remove material when said surgical cutting tool is rotated by the powered surgical handpiece, wherein said protuberances are uniformly distributed about the entire outer surface to facilitate even removal of the material when rotated by the powered surgical handpiece, said plurality of protuberance defining at least one cutting edge.
XXXII. The surgical cutting tool as set forth in clause XXXI, wherein said protuberances extend a uniform height from said outer surface.
XXXIII. The surgical cutting tool as set forth in clause XXXI, wherein said protuberances being uniformly distributed about the entire outer surface is further defined as said protuberances being uniformly spaced from one another along the entire outer surface.
XXXIV. The surgical cutting tool as set forth in clause XXXI, wherein said protuberances comprise at least a first set protuberances each extending a first uniform height from said outer surface and a second set of protuberances each extending a second uniform height from said outer surface, with said first uniform height differing from said second uniform height.
XXXV. The surgical cutting tool as set forth in clause XXXI, wherein said protuberances are aligned in at least one row, with said row helically disposed along said body of said cutting head.
XXXVI. The surgical cutting tool as set forth in clause XXXI, wherein said outer surface of said body of said cutting head has a substantially spherical configuration.
XXXVII. The surgical cutting tool as set forth in clause XXXI, wherein each of said protuberances has an arcuate configuration and extend from said arcuately configured outer surface of said body.
XXXVIII. The surgical cutting tool as set forth in clause XXXI, wherein the arcuate configuration of said protuberances is further defined as a conical configuration.
XXXIX. The surgical cutting tool as set forth in clause XXXI, wherein each of said protuberances have a uniform size and shape.
XL. The surgical cutting tool as set forth in clause XXXI, wherein each of said protuberances are disposed along said body at varying angles relative to one another.
XLI. The surgical cutting tool as set forth in clause XXXI, wherein said body and said plurality of protuberances are integrally formed.
XLII. The surgical cutting tool as set forth in clause XXXI, wherein said body and said plurality of protuberances are fused with one another and each consist of a metallic material.
XLIII. The surgical cutting tool as set forth in clause XXXI, wherein said body and said plurality of protuberances are integrally formed from deposited layers of a metallic material.
XLIV. The surgical cutting tool as set forth in clause XXXI, wherein said body and said plurality of protuberances are integrally formed from deposited, compacted, and heated layers of a metallic material.
XLV. The surgical cutting tool as set forth in clause XXXI, wherein said cutting head is free of nickel.
XLVI. The surgical cutting tool as set forth in clause XXXI, wherein said plurality of protuberance is further defined as at least twenty protuberances.
XLVII. The surgical cutting tool as set forth in clause XXXI, wherein said at least one cutting edge is further defined as at least fifty distinct cutting edges.
XLVIII. The surgical cutting tool as set forth in clause XXXI, wherein said metallic material comprises a titanium alloy.
XLIX. The surgical cutting tool as set forth in clause XXXI wherein said metallic material comprises a carbide alloy.
L. A method of manufacturing a surgical cutting tool for use with a powered surgical handpiece, the surgical cutting tool comprising a shaft extending longitudinally between a proximal end and a distal end and a cutting head mounted to the shaft at the distal end, with the cutting head comprising a body comprising an outer surface; and a plurality of protuberance each extending outwardly from the body, said method comprising the steps of: depositing layer upon layer of a metallic material to form the body of the cutting head with the outer surface having an arcuate configuration; and depositing layer upon layer the metallic material to form the plurality of protuberance of the cutting head in an arcuate configuration.
LI. The method as set forth in clause L, further comprising the step of depositing layer upon layer the metallic material to form the shaft.
LII. The method as set forth in clause L, wherein the metallic material is further defined as a titanium alloy.
LIII. The method as set forth in clause L, further comprising the step of depositing a titanium nitride coating on the titanium alloy.
LIV. The method as set forth in clause L, wherein the step of depositing layer upon layer the metallic material to form the body with the outer surface having an arcuate configuration is further defined as depositing layer upon layer the metallic material to form the body with the outer surface having a substantially spherical configuration.
LV. The method as set forth in clause L, wherein the step of depositing layer upon layer the metallic material to form the plurality of protuberance of the cutting further comprises forming the plurality of protuberance of the cutting head extending a uniform height from the outer surface and uniformly spaced from one another along the entire outer surface.
LVI. The method as set forth in clause L, wherein the step of depositing layer upon layer the metallic material to form the plurality of protuberance of the cutting further comprises forming the plurality of protuberance each having a uniform size and shape.
LVII. The method as set forth in clause L, wherein the step of depositing layer upon layer the metallic material to form the plurality of protuberance of the cutting head in an arcuate configuration is further defined as depositing layer upon layer the metallic material to form the plurality of protuberance of the cutting head in a conical configuration.
LVIII. A method of manufacturing a surgical cutting tool, said method comprising the steps of: depositing layer upon layer of a metallic material to form a body of a cutting head of the surgical cutting tool with an outer surface; and depositing layer upon layer the metallic material to form a plurality of protuberance of the cutting head.
LIX. The method as set forth in clause LVIII, wherein the protuberances are further defined as teeth.
LX. The method as set forth in clause LVIII, wherein the surgical cutting tool is further defined as a hollow tube for use with a surgical shaver.
LXI. The method as set forth in clause LVIII, wherein the surgical cutting tool is further defined as a saw blade.
LXII. A method of using a high speed drill on a patient, the high speed drill including a body defining an outer surface having an arcuate configuration, and a plurality of protuberances extending from the body, wherein the protuberances are integral with and formed from the same material as the body, and extending from the body at a distance ranging from 0.1 to 0.5 millimeters, the method comprising: rotating the body at a speed in excess of 60,000 rpm; and contacting bone and/or cartilage adjacent a critical structure with at least one of the plurality of protuberances.

1. A method of manufacturing a surgical cutting bur, the method comprising the steps of:
providing a powdered metal alloy;
sintering the powdered metal alloy and forming a unitary body portion with a plurality of protuberances extending therefrom, wherein the unitary body portion comprises a coupling region defining an interior;
removing unsintered powdered metal alloy from the interior;
positioning a drive shaft at least partially within the interior; and
coupling the drive shaft to the unitary body portion.

2. The method of claim 1, wherein the powdered metal alloy comprises stainless-steel.

3. The method of claim 1, wherein the powdered metal alloy comprises titanium.

4. The method of claim 1, wherein the step of coupling the drive shaft to the unitary body portion is further defined as bonding the drive shaft to the unitary body portion.

5. The method of claim 4, wherein bonding the drive shaft to the unitary body portion comprises a brazing process.

6. A surgical cutting bur for use with a powered surgical handpiece, the surgical cutting bur comprising:
a shaft extending longitudinally between a proximal end configured to be coupled to the powered surgical handpiece and a distal end;
a cutting head coupled to the shaft at the distal end, the cutting head comprising:
a body comprising an outer surface having an arcuate configuration; and
a plurality of protuberances each extending outwardly from the body and configured to engage and remove material when the surgical cutting bur is rotated by the powered surgical handpiece, each of the plurality of protuberance defining at least one cutting edge, and wherein the cutting head is free of cutting flutes.

7. The surgical cutting bur of claim 6, wherein the shaft is formed separately from the cutting head.

8. The surgical cutting bur of claim 7, wherein the shaft is formed from a different material than the cutting head.

9-10. (canceled)

11. The surgical cutting bur of claim 6, wherein the plurality of protuberances comprises at least a first set protuberances each extending a first uniform height from the outer surface and a second set of protuberances each extending a second uniform height from the outer surface, with the first uniform height differing from the second uniform height.

12. The surgical cutting bur of claim 6, wherein the plurality of protuberances is aligned in at least one row, with the at least one row radially arranged along the body of the cutting head.

13. The surgical cutting bur of claim 6, wherein the plurality of the protuberances extends from the unitary body portion by a distance between 0.1 mm and 0.5 mm.

14. The surgical cutting bur of claim 6, wherein the plurality of protuberances has an angled configuration and extends from the arcuately configured outer surface of the body.

15. The surgical cutting bur of claim 14, wherein the angled configuration of the plurality of protuberances is further defined as a frustopyramidal configuration.

16-18. (canceled)

19. The surgical cutting bur of claim 6, wherein the body and the plurality of protuberances are fused together, and each comprises a metallic material.

20. The surgical cutting bur of claim 19, wherein the body and the plurality of protuberances are integrally formed from deposited layers of the metallic material.

21. The surgical cutting bur of claim 19, wherein the body and the plurality of protuberances are integrally formed from deposited, compacted, and heated layers of the metallic material.

22. The surgical cutting bur of claim 21, wherein the metallic material comprises a carbide alloy.

23. The surgical cutting bur of claim 19, wherein the metallic material comprises a titanium alloy.

24. The surgical cutting bur of claim 19, wherein the metallic material comprises a stainless-steel alloy.

25. The surgical cutting bur of claim 6, wherein the cutting head is free of nickel.

26-27. (canceled)
